(19) United States
(12) Patent Application Publication
(10) Pub. No.: US 2014/0005568 A1
(43) Pub. Date: Jan. 2, 2014

(54) SENSORY TESTING SYSTEMS, METHODS AND DEVICES

(71) Applicant: Todd O'Brien, Orono, ME (US)

(72) Inventor: Todd O'Brien, Orono, ME (US)

(21) Appl. No.: 13/903,284

(22) Filed: May 28, 2013

Related U.S. Application Data
(60) Provisional application No. 61/665,693, filed on Jun. 28, 2012.
(60) Provisional application No. 61/665,710, filed on Jun. 28, 2012.

(51) Int. Cl. A61B 5/00

(52) U.S. Cl. CPC A61B 5/4827
USPC 600/557

(57) ABSTRACT

The present invention relates to a hand-held instrument used in the assessment of cutaneous tactile sensation. The device consists of a shape memory alloy wire with selected bending force rating and an integral force indicator mechanism. The force indicator is comprised of a wire attachment means, spring-mounted battery and a light source. A hand-held instrument used in the assessment of cutaneous tactile sensation, specifically the ability to discriminate between sharp and dull stimuli, is disclosed. The device consists of two handle-mounted monofilaments with calibrated bending force ratings; one terminating in a sharp tip, the other in a dull tip. Users apply each monofilament separately to the skin thereby transmitting either a sharp or dull stimuli at a specific force. The accuracy of feedback to these stimuli will alert users to the presence or absence of protective sensation. Methods of use and kits are also disclosed.

### BACKGROUND OF THE DISCLOSURE

1. Field of Invention

The present invention pertains to a shape memory wire sensory tester with force indicator. More particularly, the present invention relates to a method of assuring proper force application to the skin during sensation testing. The device includes a shape memory wire and a force indicator mechanism. The wire when pressed against the skin applies a selected force at its bending point. A light source illuminates signaling proper force application to the skin. Failure of the light source to illuminate indicates less than appropriate force application to the skin. This alerts the user to the necessity of monofilament replacement. Additionally, the wire may be refreshed between uses through application of an electrical current. The resulting heating of the wire facilitates a phase transformation which restores its original configuration.

2. Background of the Invention

Monofilaments have been a simple and popular method of assessing cutaneous sensation for many years. This method of sensory testing was refined by J. Semmes and S. Weinstein who in the 1950's developed a series of nylon monofilaments, which were pre-calibrated by virtue of their diameter. These monofilaments applied specific force levels to the skin allowing the user to assess the threshold of cutaneous sensation with more accuracy than had been possible with previous instrumentation. This approach proved valuable in identifying patients with sensory deficits, which in many cases predispose them to cutaneous injury such as ulceration. A specific area where monofilament testing has gained widespread support pertains to diabetics. It is known that diabetics tend to develop neuropathy in their feet. This neuropathy can lead to ulceration and subsequent infection. A cascade of events leading to lower extremity amputation and even death has been well defined in this patient population.

### FIELD OF THE DISCLOSURES

A variety of investigators have documented the value of monofilament testing in the diabetic patient. The ability of the patient to perceive a specific amount of force on the skin has been used as a method of identifying those at risk for future foot ulceration. In particular, the inability to perceive ten grams of force has been established as a useful threshold of cutaneous sensation in predicting those at risk for ulceration. It has also been shown that commercially available monofilaments do not consistently provide this desired level of force to the skin.

### Prior Art

Christy was granted U.S. Pat. No. 5,823,969 on Oct. 20, 1998 for a tactile sensory testing instrument. This device allowed the user to apply a monofilament to the skin in a perpendicular manner. The monofilament folded into an integral handle thereby protecting it. Linden was issued U.S. Pat. No. 6,234,976 on May 22, 2001 for a device for evaluating protective sensation. This device also provided a monofilament with integral handle very similar to the device taught in Christy's U.S. Pat. No. 5,823,969. Christy was issued an additional U.S. Pat. No. 6,234,977 on May 22, 2001 for a variable-force monofilament sensory device and methods of using same. This device sought to overcome some of the problems with previous designs by incorporating a nitinol wire instead of a nylon monofilament. Nitinol wire is not subject to the same changes in force delivery due to humidity or temperature variations. The device also disclosed a method of using a variety of interchangeable tip members providing specific force to the skin.

The prior art has thus far failed to contemplate a method of indicating appropriate force delivery to the skin surface. Indication of appropriate force transmittal will ensure identification of at risk patients thereby improving the quality of medical care. The prior art also fails to take advantage of the Martensitic phase transformation properties of shape memory alloys such as nitinol. This quality when utilized will permit restoration of a nitinol wire's original configuration and bending force resulting in a more accurate and durable sensory testing instrument.

### SUMMARY OF THE DISCLOSURE

According to embodiments, there is provided a hand-held instrument used for evaluation of cutaneous sensory perception comprised of a handle, a shape memory alloy wire with a preselected bending force, a force indicator with shape memory alloy wire attachment means further comprised of a precalibrated spring-mounted battery and illumination source, and, a finger-actuated means of applying electrical current to the shape memory alloy wire.

According to embodiments, there is provided a device wherein when the user applies the shape memory alloy wire to the skin in a perpendicular manner it provides a preselected force at its bending point.

According to embodiments, there is provided a device of claim one wherein when a threshold force is transmitted to the skin, the spring-mounted battery contacts a light source thereby illuminating and signaling proper force application to the user.

Briefly stated, the present disclosure provides users with a a hand-held instrument used in the assessment of cutaneous tactile sensation. The device consists of a shape memory alloy wire with selected bending force rating and an integral force indicator mechanism. The force indicator is comprised of a wire attachment means, spring-mounted battery and a light source. During use the wire transmits a selected force at its bending point to a spring-mounted battery. This force moves the battery into contact with the light source, subsequently causing it to illuminate. Failure of the light force to illuminate, signals insufficient force transmittal by the monofilament. This would alert the user of the need for monofilament replacement. This mechanism assures proper force application to the skin thereby resulting in more accurate sensation testing. Alternatively, a strain gauge could be used in place of the spring mechanism to achieve the same result. An additional feature of this invention is the application of heat to the shape memory wire thereby reconstituting the original configuration and bending force between user applications.

### BRIEF DESCRIPTION OF THE DRAWINGS

FIG. 1 is a schematic view of the device in an inactive state.

FIG. 2 is a view of the device as used in relation to a body part to be tested.

FIG. 3 shows a schematic of the present invention;

FIG. 4 shows a schematic of the present invention:

FIG. 5 schematically illustrates embodiments according to the instant teachings; and

FIG. 6 schematically illustrates embodiments according to the instant teachings.

### DETAILED DESCRIPTION OF THE DISCLOSURE

It is an object of the present invention to provide a shape memory wire sensory tester with force indicator. The device will illuminate upon transmittal of a specific load to the skin. This informs the user that the shape memory wire is providing the appropriate load. Failure of the device to illuminate signals the need for wire replacement.

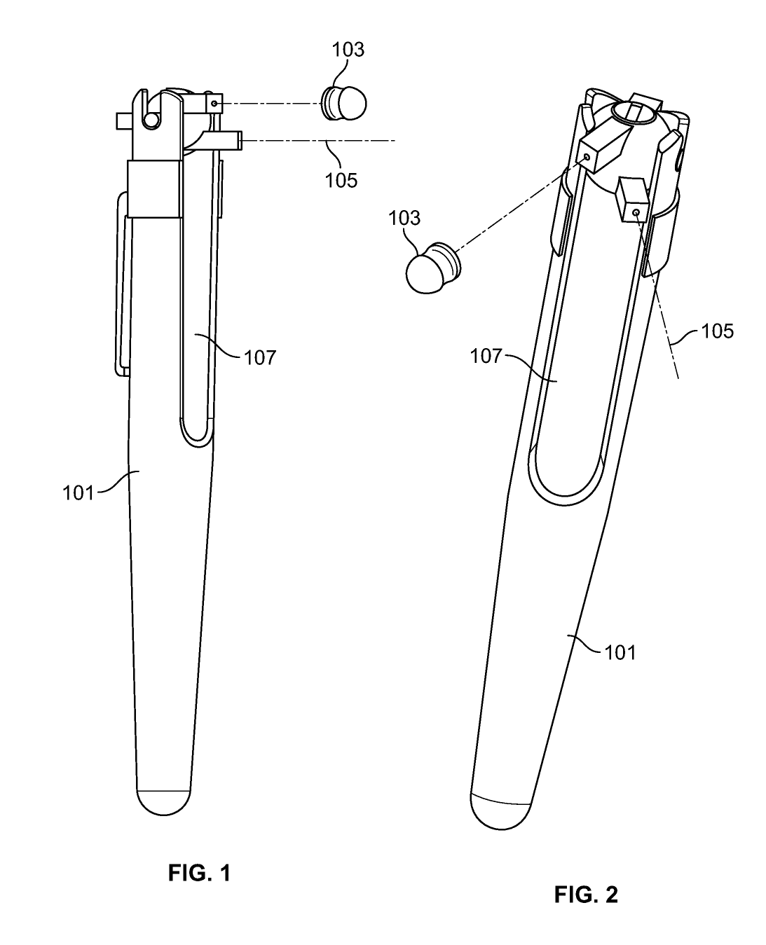

It is another object of the present invention to utilize the phase transformation capabilities of nitinol wire to restore its original shape and bending force thereby ensuring more accurate sensation testing (see FIGS. 1 and 2).

It is an object of the present invention to provide a calibrated sharp/dull discriminator for assessment of cutaneous sensation.

According to embodiments, there is disclosed a hand-held instrument used to assess cutaneous tactile sensation.

According to embodiments, a method of applying calibrated for the sign during sharp/dull discrimination.

Briefly stated, the present disclosure provides users with a hand-held instrument used in the assessment of cutaneous tactile sensation, specifically the ability to discriminate between sharp and dull stimuli. The device consists of two handle-mounted monofilaments with calibrated bending force ratings; one terminating in a sharp tip, the other in a dull tip. Users apply each monofilament separately to the skin thereby transmitting either a sharp or dull stimuli at a specific force. The accuracy of feedback to these stimuli will alert users to the presence or absence of protective sensation.

### DETAILED DESCRIPTION OF THE DISCLOSURE

The present inventor has discovered more efficient ways to test a patient's ability to perceive sharp and dull sensations.

References to the current disclosure are described in the table below, and each of these patents is expressly incorporated by reference, along with U.S. Pat. Nos. 4,665,906; 5,067,957; 5,190,586; and 5,597,378:

[symbol]

### DETAILED DESCRIPTION OF THE FIGURES

FIG. 1 shows a schematic of the present invention;

FIG. 2 shows a schematic of the present invention;

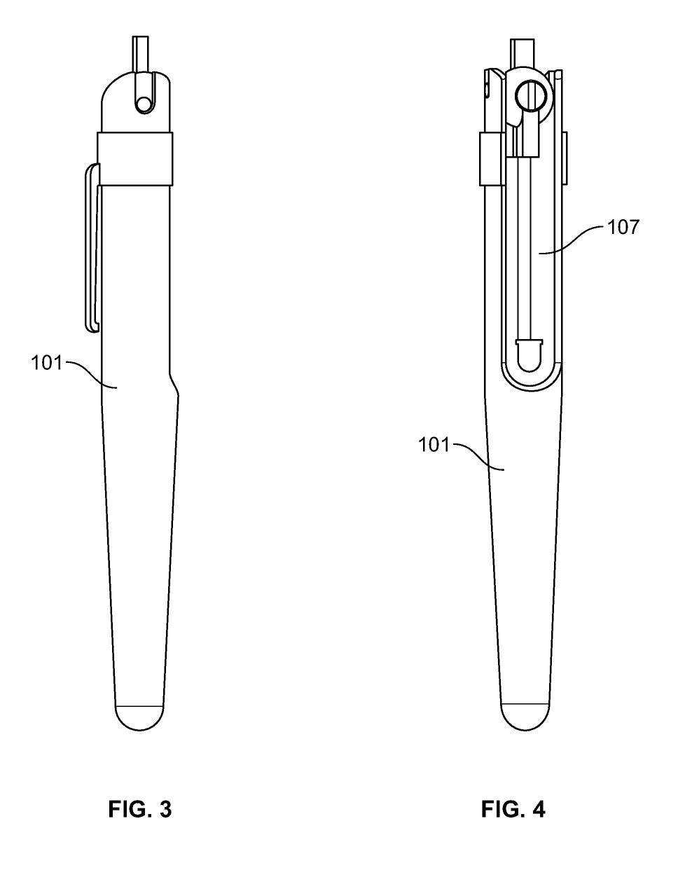

FIG. 3 shows a schematic of the present invention;

FIG. 4 shows a schematic of the present invention;

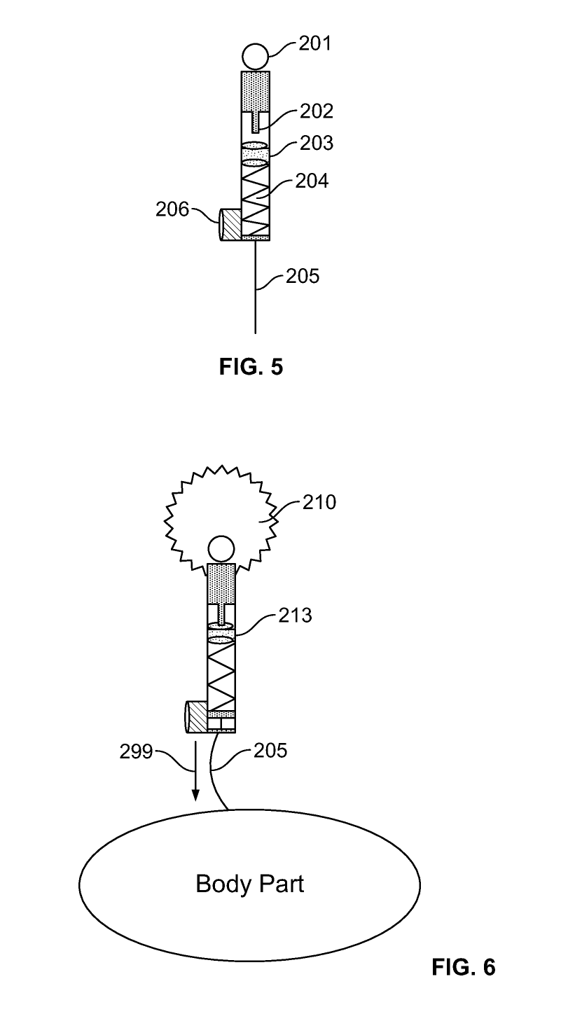

FIG. 5 schematically illustrates embodiments according to the instant teachings; and

FIG. 6 schematically illustrates embodiments according to the instant teachings;

Referring now to FIGS. 1-4, FIG. 1 shows an open configuration, side-view of Apparatus 101, having monofilaments 103 (dull) and 105 (sharp), along with recess 107. FIG. 2 shows a front/partial plan view with the same elements, while FIG. 3 and FIG. 4 show closed configurations from the front and side, respectively.

Referring to FIG. 5, disclosed is an embodiment with LED indicator on/off in an OFF state, 201 LED contact point 202, operatively linked to battery 203, spring 204 and shape memory wire 205. Wire refresh control button 206 imparts electrical current to wire 205.

FIG. 6 shows LED wire in an On state 210, battery 203 making contact with LED contact point 213, and shape memory wires 205 bending at 10 grams of applied force 299, when impacting a body part.

Those skilled in the art readily understand the novelty and inherent methods, systems and related devices disclosed according to the instant teachings, thus further details are omitted at this time, as known to artisans.

While the method and apparatus have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

It should also be understood that a variety of changes may be made without departing from the essence of the invention. Such changes are also implicitly included in the description. They still fall within the scope of this invention. It should be understood that this disclosure is intended to yield a patent covering numerous aspects of the invention both independently and as an overall system and in both method and apparatus modes.

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these.

Particularly, it should be understood that as the disclosure relates to elements of the invention, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this invention is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Random House Webster's Unabridged Dictionary, latest edition are hereby incorporated by reference.

Finally, all references listed in the Information Disclosure Statement or other information statement filed with the application are hereby appended and hereby incorporated by reference; however, as to each of the above, to the extent that such information or statements incorporated by reference might be considered inconsistent with the patenting of this/these invention(s), such statements are expressly not to be considered as made by the applicant.

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC 132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, the use of the transitional phrase “comprising” is used to maintain the “open-end” claims herein, according to traditional claim interpretation. Thus, unless the context requires otherwise, it should be understood that the term “comprise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

1. A method of testing sensory perception in peripheral nerves, comprising, in combination:
providing apparatus having a handle-member at least sharp and dull monofilaments of Nitinol®;
a recess for housing the same; and,
actuating means;
extending the Nitinol® monofilaments into an orthogonal position relative to the long axis of the apparatus; and,
applying pressure to a patient's dermal layer;
whereby calibration and reproducibility of said pressure application are housed, sorted, and utilized.

2. The method, according to claim 1, wherein the Nitinol's® shape-memory alloy features are used for calibration and pressure monitoring.

3. The method of claim 2, further comprising transitioning from Austenite to Martensite as phase changing.

4. A hand-held instrument used for evaluation of cutaneous sensory perception, which comprises, in combination:
a handle;
a shape memory alloy wire with a preselected bending force;
a force indicator with shape memory alloy wire attachment means further comprised of a precalibrated spring-mounted battery and illumination source; and,
a finger-actuated means of applying electrical current to the shape memory alloy wire.

5. The device of claim 4, wherein the user applies the shape memory alloy wire to the skin in an orthagonal manner to provide a preselected force at its bending point.

6. The device of claim 5, wherein when a threshold force is transmitted to the skin, the spring-mounted battery contacts a light source thereby illuminating and signaling proper force application to the user.

7. The device of claim 6, wherein Martensitic phase transformation is used to generate the preselected bending force.

8. The device of claim 7, wherein said instrument can be managed by one hand of a user.

9. The device of claim 4, wherein angles other than orthogonal are utilized.

10. The device of claim 8, wherein only patient-appropriate force is delivered, permitting restoration.

11. The device of claim 9, wherein only patient-appropriate force is delivered, permitting restoration.

12. A kit, comprising in combination, the device of claim 8, and instructions for use.

13. A kit, comprising in combination, the device of claim 9, and instructions for use.
